(19) United States
(12) Patent Application Publication
(10) Pub. No.: US 2006/0105341 A1
(43) Pub. Date: May 18, 2006

(54) Trap-tagging: a novel method for the identification and purification of rna-protein complexes

(76) Inventors: Henry M Krause, Mississauga (CA); Andrew J Simmonds, Edmontoo (CA)

Correspondence Address: CONLEY ROSE, P.C., P. O. BOX 3267, HOUSTON, TX, 77253-3267, US

(21) Appl. No.: 10/531,095

(22) PCT Filed: Oct. 10, 2003
(86) PCT No.: PCT/CA03/01555

§ 371 Date: Apr. 7, 2005

(30) Foreign Application Priority Data
Oct. 11, 2002 (CA) 2407825

(51) Int. Cl. C12Q 1/68; C07H 21/02; C12P 21/06; C07H 21/04

(52) U.S. Cl. 435/6; 530/352; 435/69.1; 435/320.1; 435/325; 536/23.1

(57) ABSTRACT

Conventional methods for the isolation and identification of specific RNA-protein complexes are plagued by a number of problems not encountered in genomics or proteomics. Here we describe a two step affinity purification method used to isolate RNA-protein complexes. The TRAP (Tandem RNA Affinity Purification) tag is a dual RNA tagging system that facilitates gentle purification of RNA molecules along with the proteins, RNAs and other small molecules specifically associated with them.

### FIELD OF INVENTION

This invention relates to a method for the identification and purification of RNA-protein complexes formed in vivo and in vitro.

### BACKGROUND

In addition to serving as essential intermediates between genes and proteins, RNA molecules also serve structural and regulatory roles in a rapidly growing list of biological processes. These include all of the basic steps of transcription initiation, splicing, localization, translation and stability (Dreyfuss, et al., 2002; Szymanski et al., 2003; Doudna and Rath, 2002; Erdmann et al., 2001; Pesole et al., 2001; Berkhout et al., 1989) as well as processes such as dosage compensation (Bell et al., 1988; Lee and Jaenisch, 1997; Meller et al., 2000; Salido et al., 1992), heterochromatin formation (Lee et al., 1997) and, telomere maintenance (Le et al., 2000). Importantly, the genomes of many viruses are encoded as RNA rather than DNA, and much of their infective cycles are controlled by RNA biochemistry (Berkhout et al., 1989), as are the host defense systems that block the infection process (Mahalingam et al., 2002). Clearly, these molecules and processes are crucial for cell and pathogen viability, and are excellent targets for drug intervention.

The recently coined term ribonomics has been defined as a complete understanding of mRNA metabolism, structure, interactions and function (Keene 2001; Tenenbaum et al., 2000). A comprehensive cataloguing of all ribonucleoprotein (RNP) complexes is an essential aspect of this major endeavor. However, the methodologies currently employed to identify RNA associated molecules are not ideally suited for such an endeavor. For example, RNA binding proteins generally do not have the same specificity as DNA binding proteins. Consequently, techniques that identify individual RNA-protein interactions frequently isolate proteins that are irrelevant to the processes being studied. Indeed, there is increasing evidence that many high affinity RNA/protein interactions require multiple contacts between cis-acting elements and several proteins within a complex (Chartrand et al., 2001). This complexity has several deleterious effects for the detection of interactions in vitro. First, if individual interactions are weak, they may not occur in vitro. Second, if pre-formed multimeric complexes are stable, individual components may not be available for de novo assembly.

This would lead to the isolation of other more available and abundant molecules that are irrelevant to the process being investigated.

A method capable of isolating specific RNA-protein complexes that form in vivo would circumvent many of the above-listed problems. If a similar method could be used to analyze complexes in vitro, the similarity between the results would indicate whether or not the in vivo process was required to study the RNA-protein complex in question.

### SUMMARY

The invention provides a method for purifying an RNA-protein complex formed in vitro comprising providing an RNA fusion molecule comprising a target RNA sequence and at least two different RNA tags, wherein at least one RNA tag interacts with a ligand in a reversible manner; contacting the RNA fusion molecule with a cellular extract; providing conditions that allow the formation of an RNA-protein complex on the target RNA sequence; and subjecting the RNA-protein complex to at least two different affinity purification steps, each step comprising binding one RNA tag to an affinity resin capable of selectively binding one RNA tag and eluting the RNA tag from the affinity resin after substances not bound to the affinity resin have been removed. In one embodiment the RNA fusion molecule is contacted with a protein mixture in place of a cellular extract.

The invention also provides for a method for purifying an RNA-protein complex formed in vivo comprising: expressing in a eukaryotic cell an RNA fusion molecule comprising a target RNA sequence and at least two different RNA tags, wherein at least one RNA tag interacts with a ligand in a reversible manner; providing conditions that allow the formation of an RNA-protein complex on the target RNA sequence; generating a cellular extract; subjecting the cellular extract to at least two different affinity purification steps, each step comprising binding one RNA tag to an affinity resin capable of selectively binding one RNA tag and eluting the RNA tag from the affinity resin after substances not bound to the affinity resin have been removed.

The invention also provides for a protein identified by isolating an RNA-protein complex formed in vitro or in vivo according to the methods of the current invention.

In one embodiment, at least one RNA tag binds to an affinity resin through a fusion protein comprising a polypeptide that binds specifically to the RNA tag and a polypeptide that binds specifically to the affinity resin. In a preferred embodiment, the polypeptide that binds specifically to the affinity resin is selected from the group consisting of a maltose binding protein, a 6-histidine peptide, glutathione S transferase and a portion thereof sufficient to bind specifically to the affinity resin.

Another aspect of the invention is an RNA fusion molecule comprising a target RNA sequence and at least two different RNA tags, wherein at least one RNA tag interacts with a ligand in a reversible fashion.

In one embodiment of the present invention at least one of the RNA tags is repeated. In a preferred embodiment the RNA tags are selected from streptavidin binding sequence (S1), an MS2 coat protein binding sequence, a streptomycin binding sequence (Streptotag), a sephadex binding sequence (D8), an N protein binding sequence (nut), a REV binding sequence, a TAT-binding sequence and an R17 coat protein binding sequence. In yet another preferred embodiment the RNA tags are at least one MS2 coat protein binding sequence and at least one streptavidin binding sequence. In a most preferred embodiment the RNA tags are six MS2 coat protein binding sequences and two streptavidin binding sequences.

In another embodiment of the current invention, the RNA fusion molecules further comprise at least one insulator sequence.

The invention also provides for isolated DNA constructs encoding the RNA fusion molecules of the present invention and for vectors and host cells expressing the isolated DNA constructs.

The invention relates to a method for screening test compounds or proteins for their ability to modulate or regulate an RNA-protein complex by performing the methods of the present invention for purifying RNA-protein complexes formed in vitro or in vivo and observing a difference, if any, between the RNA-protein complexes purified in the presence of the test compounds or proteins and the absence of the test compounds or proteins, wherein a difference indicates that the test compounds or proteins modulate the RNA-protein complex. This invention provides an isolated DNA construct comprising a transcription cassette, which comprises a promoter sequence, a bait sequence operably linked to the promoter, a transcriptional termination sequence which comprises a stop signal for RNA polymerase and a polyadenylation signal for polyadenylase, and at least two tag sequences.

In one embodiment the isolated DNA construct comprises at least one streptavidin binding sequence [SEQ ID NO:1 SEQ ID NO:2 SEQ NO 17] and at least one MS2 coat protein binding sequence [SEQ ID NO:4, SEQ ID NO:6 SEQ ID NO:7 SEQ NO 18]. In yet another embodiment, the isolated DNA construct comprises at least one tag sequence which hybridizes to the streptavidin binding sequence [SEQ ID NO:2] and at least one tag sequence which hybridizes to the MS2 coat protein sequence [SEQ ID NO:4] under high stringency hybridization conditions.

The invention also provides an isolated DNA construct comprising a transcription cassette, which construct comprises, a promoter sequence, a bait sequence operably linked to the promoter, a transcriptional termination sequence, which comprises a stop signal for RNA polymerase and a polyadenylation signal for polyadenylase; and at least three tag sequences. In another embodiment the isolated DNA construct comprises at least one streptavidin binding sequence [SEQ ID NO:2 SEQ NO 17] and at least two MS2 coat protein binding sequences [SEQ ID NO:7 SEQ NO 18]. In yet another embodiment the isolated DNA construct at least one tag sequence which hybridizes to the streptavidin binding sequence [SEQ ID NO:2 SEQ NO 17] and at least two tag sequences which hybridize to the MS2 coat protein sequence [SEQ ID NO:7 SEQ NO 18] under high stringency hybridization conditions.

In one embodiment, the isolated DNA constructs further comprise at least three insulator sequences, and in another embodiment at least four insulator sequences.

The present invention relates to expression vectors and host cells comprising the isolated DNA constructs.

Another aspect of the invention is an RNA fusion molecule comprising a target RNA sequence and at least two RNA tags, wherein at least one of the RNA tags interacts with a ligand in a reversible fashion. In one embodiment the RNA fusion molecule comprises at least one streptavidin binding tag [SEQ ID NO:3] and at least one MS2 coat protein binding tag [SEQ ID NO:5].

The current invention also relates to an RNA fusion molecule comprising a target RNA sequence and at least three RNA tags, wherein at least two of the RNA tags interact with a ligand in a reversible fashion. In another embodiment, the RNA fusion molecule comprises at least one streptavidin binding tag [SEQ ID NO:3] and at least two MS2 coat protein binding tags [SEQ ID NO:8].

In one embodiment, the RNA fusion molecules further comprise at least 3 insulators, and in another embodiment, 4 insulators.

The invention provides a method for isolating an RNA-protein complex formed in vivo comprising, expressing in a eukaryotic cell an RNA fusion molecule of the current invention, generating a whole cell extract, passing the extract over a first solid support comprising streptavidin protein, eluting a first eluate with the addition of biotin, collecting the first eluate, passing the first eluate over a second solid support comprising MS2 coat protein, eluting a second elute with the addition of a reagent selected from the group consisting of glutathione, RNAse or a denaturant, and collecting the second elute, wherein the second eluate contains the isolated RNA-protein complex.

The current invention provides a method of identifying a protein in an RNA-protein complex comprising isolating an RNA-protein complex formed in vivo comprising, expressing in a eukaryotic cell an RNA fusion molecule of the current invention, generating a whole cell extract, passing the extract over a first solid support comprising streptavidin protein, eluting a first eluate with the addition of biotin, collecting the first eluate, passing the first eluate over a second solid support comprising MS2 coat protein, eluting a second elute with the addition of a reagent selected from the group consisting of glutathione, RNAse or a denaturant, and collecting the second elute, wherein the second eluate contains the isolated RNA-protein complex and identifying the protein in the RNA-protein complex.

The invention also provides for a protein identified by performing the methods of isolating an RNA-protein complex formed in vivo.

Another aspect of the current invention is a method for isolating an RNA-protein complex formed in vitro comprising, (a) expressing a RNA fusion molecule of the current invention in vitro, (b) obtaining a whole cell extract, (c) passing the whole cell extract over a first solid support comprising streptavidin protein, (d) eluting a first eluate with the addition of biotin, (e) collecting the first eluate, (f) passing the first eluate over a second solid support comprising MS2 coat protein, (g) eluting a second elute with the addition of a reagent selected from the group consisting of glutathione, RNAse or a denaturant, and (h) collecting the second eluate, wherein the second eluate contains the isolated RNA-protein complex. In one embodiment steps (c) to (e) are repeated.

The current invention provides a method of identifying a protein in an RNA-protein complex comprising isolating an RNA-protein complex formed in vitro comprising (a) expressing a RNA fusion molecule of the current invention in vitro, (b) obtaining a whole cell extract, (c) passing the whole cell extract over a first solid support comprising streptavidin protein, (d) eluting a first eluate with the addition of biotin, (e) collecting the first eluate, (f) passing the first eluate over a second solid support comprising MS2 coat protein, (g) eluting a second elute with the addition of a reagent selected from the group consisting of glutathione, RNAse or a denaturant, and (h) collecting the second eluate, wherein the second eluate contains the isolated RNA-protein complex and identifying the protein in the RNA-protein complex. In one embodiment, steps (c) to (e) are repeated.

The invention also provides for a protein identified by the methods of isolating an RNA-protein complex formed in vitro.

The invention also relates to a method of screening for a compound that modulates the formation of an RNA-protein complex formed in vivo comprising, expressing in a eukaryotic cell an RNA fusion molecule of the current invention in the presence of a test compound, generating a whole cell extract, passing the extract over a first solid support comprising streptavidin protein, eluting a first eluate with the addition of biotin, collecting the first eluate, passing the first eluate over a second solid support comprising MS2 coat protein, eluting a second eluate with the addition or a reagent selected from the group consisting of glutathione, RNAse or a denaturant, collecting the second eluate, wherein the second eluate contains the isolated RNA-protein complex, measuring th amount of isolated RNA-protein complex present, and comparing the amount of isolated RNA-protein complex present in the absence of the compound to be tested.

The invention also provides for a method of screening for a compound that modulates the formation of an RNA-protein complex formed in vitro comprising, (a) expressing an RNA fusion molecule of the current invention in vitro, (b) obtaining a whole cell extract, (c) passing the whole cell extract over a first solid support comprising streptavidin protein, (d) eluting a first eluate with the addition of biotin, (e) collecting the first eluate, (f) passing the first eluate over a second solid support comprising MS2 coat protein, (g) eluting a second eluate with the addition of a reagent selected from the group consisting of glutathione, RNAse or a denaturant, (h) collecting the second eluate, wherein the second eluate contains the isolated RNA-protein complex, (i) measuring the amount of isolated RNA-protein complex present; and (j) comparing the amount of isolated RNA-protein complex present in the absence of the compound to be tested. In one embodiment, steps (c) to (e) are repeated. The invention also relates to the compounds or proteins that modulate the RNA-protein complexes and that are identified by the screening methods of the current invention.

The invention also provides for kits for detecting an RNA-protein complex comprising the RNA fusion molecules, the isolated DNA constructs and the vectors of the present invention.

### DETAILED DESCRIPTION OF THE DRAWINGS

Preferred embodiments of the invention will be described in relation to the drawings in which:

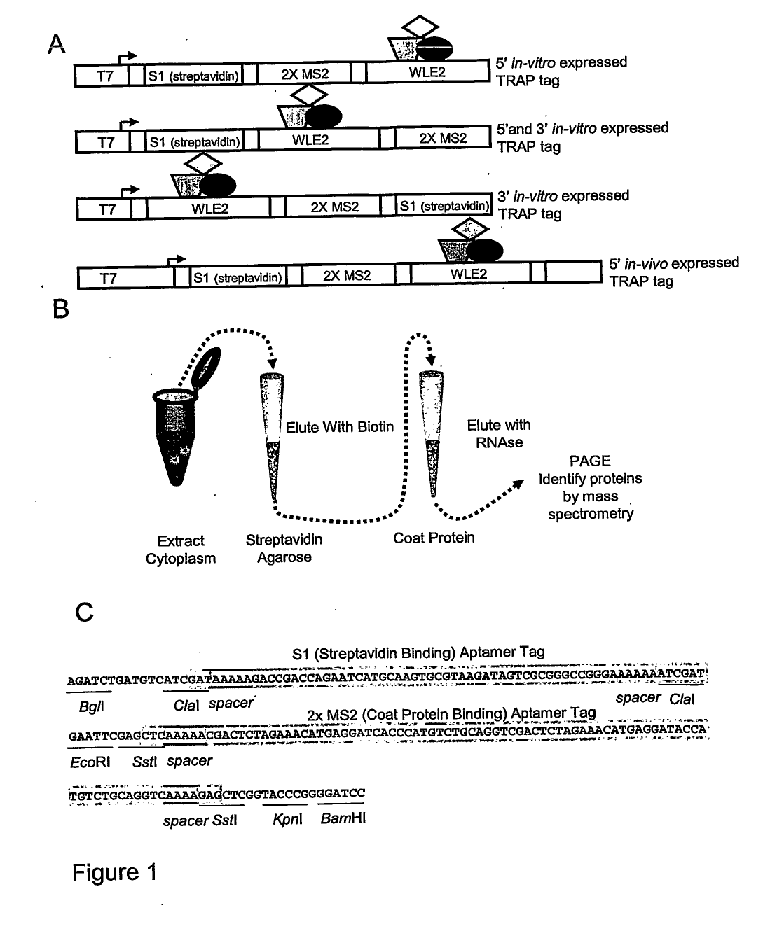

FIG. 1. Tandem RNA affinity purification. A) RNAs of interest are tagged at their 5′ or 3′ end with two different RNA tags. The tagged RNAs are then expressed either in vitro or in vivo and tested for function. B) Functional complexes containing the tagged RNA are purified from extracts using two affinity resins, each of which is capable of binding one of the tags. An important aspect of the tags, particularly the first tag used, is that it must be capable of being dissociated from its affinity resin using conditions that do not disrupt the RNA-protein complex. Proteins eluted from the second resin are generally sufficiently pure for identification by SDS PAGE, silver staining, and Mass Spectrometry. Bound RNAs can also be identified using RTPCR or microarray analysis.

C) Sequence of the TRAP cassette. Sequences in parentheses indicate each of the different functional motifs within the TRAP cassette.

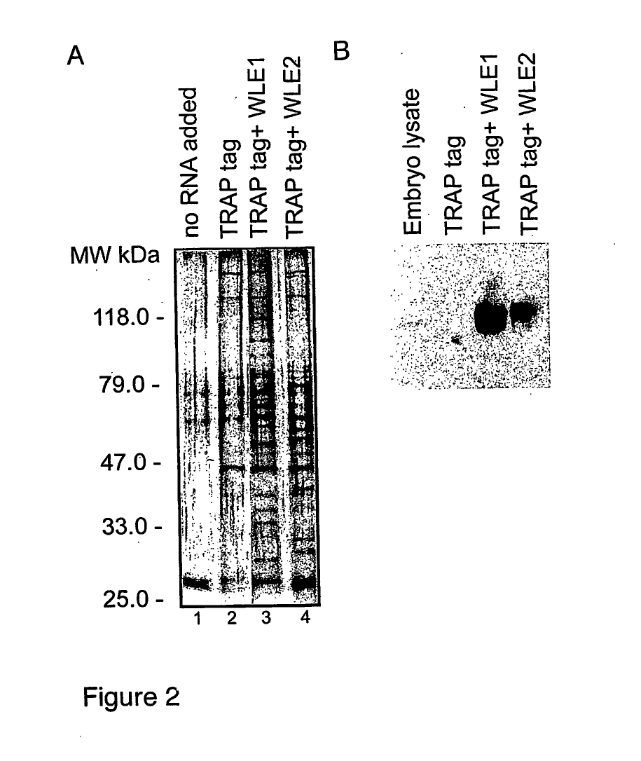

FIG. 2. TRAP-tag purification using in vitro transcribed RNA.

A) In vitro purification of proteins from extracts. Embryonic cytoplasmic extracts were mixed with TRAP-tagged RNA or untagged control RNA and purified using TRAP. Eluates were subjected to SDS PAGE and silver staining. Lane 1: no RNA added to the extract. Lane 2: No bait RNA fused to the TRAP RNA. Lane 3: purification using TRAP RNA fused to a localization element from the 3′UTR of the Drosophila wingless gene mRNA (WLE1). Lane 4: protein purification using TRAP RNA fused to a second transcript localizing element in the wingless mRNA 3′ UTR (WLE2). Note that the RNAs containing the two baits (WLE1 and WLE2) bind proteins that are not bound by the resins or TRAP RNA alone. B) In vitro purification of Bic-D from embryo extracts. Following the purification as described above, eluted proteins were subjected to SDS PAGE and then transferred to membranes for Western blotting with anti Bic-D antiserum. Lanes 2-4 are as described above. Note that the Bic-D signal is highly enriched in lanes 3 and 4 after TRAP purification with the WLE1 and WLE2 localization elements. Bic-D was detected in the crude extract (Lane 1) after much longer exposures (not shown).

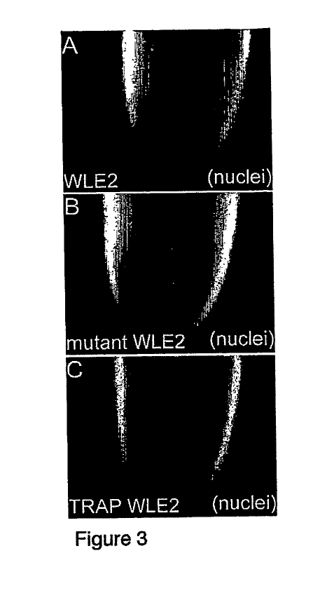

FIG. 3. Localization of TRAP-tagged WLE RNAs in Drosophila embryos.

To ensure that the TRAP-tag does not interfere with bait RNA function, WLE localization elements fused to TRAP RNAs were tested for localization activity in embryos. A) Fluorescently labeled untagged WLE2 RNA (red) moves to the apical cytoplasm above the nuclei (green) after injection into a syncitial blastoderm staged embryo. B) A mutated WLE2 element has no localizing activty. Labeled RNA remains below the nuclei. C) TRAP-tagged WLE2 RNA moves apically in the same manner as untagged WLE2 RNA, indicating that the addition of the TRAP sequence has no obvious effect on localization function.

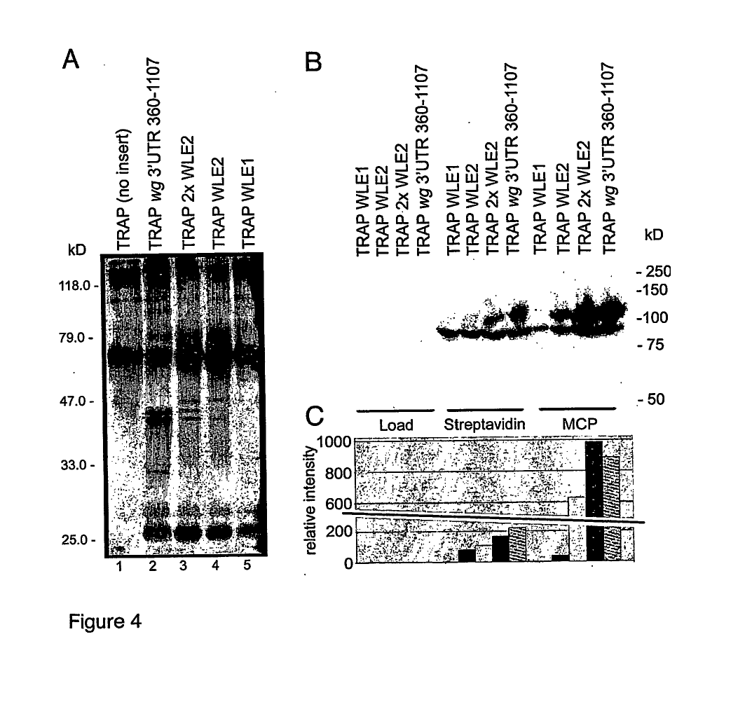

FIG. 4. In vivo TRAP

A) TRAP purification using extracts in which TRAP-tagged WLE RNAs were expressed in vivo. Lane 1: TRAP RNA with no bait; Lane 2: TRAP RNA containing a large portion of the wg 3′UTR that encompasses WLE2; Lane 3: TRAP-tag fused to a tandem duplication of WLE2; Lane 4: TRAP-tagged WLE2. Lane 5: TRAP-tagged WLE1.

B) Western blot of TRAP purified proteins with anti-Bic-D antibody. Proteins loaded in each lane were purified using the TRAP constructs listed above. Fractions from the load, streptavidin column eluate and MS2 column elute are as indicated below.

C) Quantitation of Bic-D signals. ECL-generated band intensities were measured using a phosphoimager. Values shown are relative to background.

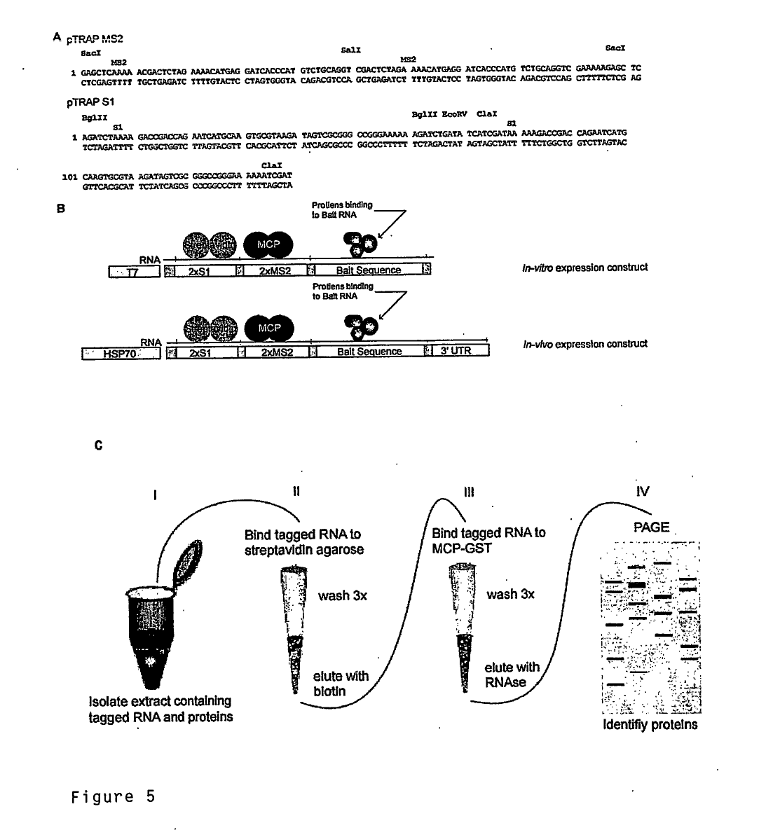

FIG. 5. Tandem RNA affinity purification.

A) TRAP cassette DNA sequence. MS2 and S1 motifs (indicated) are flanked by insulator sequences and restriction sites that facilitate the shuffling of motifs and insertion into various vectors.

B) Schematic map of the 2XS1 and 2XMS2 cassettes introduced into in vitro (top) or in vivo (bottom) expression vectors. RNAs of interest can be tagged at their 5′ or 3′ end with two different RNA tags. Tagging at 5′ end is shown here.

C) Overview of the TRAP purification procedure. For the second affinity column, elution can be achieved using RNAse (indicated), high salt, glutathione or denaturants. Alternatively, if RNA components are being identified, proteases can be used.

Table 1. Suitability of tags for TRAP-tag purification. Tags used for affinity purification are shown in the left hand column. Sizes, affinity matrices, eluting reagents, and performance are shown in the columns to the right. Binding and elution efficiencies were determined using ³²P-labeled RNAs expressed in vitro and are expressed as percentage of label loaded.

### DETAILED DESCRIPTION

The present invention will now be described more fully with reference to the accompanying drawings, in which preferred embodiments of the invention are shown. This invention may, however, be embodied in different forms and should not be construed as limited to the embodiments set forth herein. Rather, these embodiments are provided so that this disclosure will be thorough and complete, and will fully convey the scope of the invention to those skilled in the art.

The term “bait sequence” as used herein, is a cDNA or DNA sequence that encodes a target RNA sequence. Examples of suitable bait sequences include RNAs, such as, the HUV Tat-binding tar element, the E. coli N protein binding box B element, and various recognition elements within RNA splice sites.

The term “isolated DNA sequence” as described herein includes DNA whether single or double stranded. The sequence is isolated and/or purified (i.e. from its natural environment), in substantially pure or homogeneous form, free or substantially free of nucleic acid or genes of the species of interest or origin other than the promoter or promoter fragment sequence.

The DNA sequence according to the present invention may be wholly or partially synthetic.

The term “isolated” encompasses all these possibilities.

The term “operably linked” as described herein means joined as part of the same nucleic acid molecule, suitably positioned and oriented for transcription to be initiated from the promoter.

The term “promoter” as described herein refers to a sequence of nucleotides from which transcription may be initiated of DNA operably linked downstream (i.e. in the 3′ direction on the sense strand of double-stranded DNA). The promoter or promoter fragment may comprise one or more sequence motifs or elements conferring developmental and/or tissue-specific regulatory control of expression. For example, the promoter or promoter fragment may comprise a neural or gut-specific regulatory control element.

The term “DNA tag” as used herein refers to short DNA or cDNA sequences that encode a binding partner for a ligand. The ligand may be any molecule that specifically binds to the binding partner such as, antibiotics, antibodies or specific proteins. The DNA tags of the current invention may be located 3′ or 5′ to the bait sequence. DNA tags encode RNA tags.

The term “RNA tags” as used herein refers to short RNA sequences that function as a binding partner for a ligand. The RNA tags must be short, fully modular and must not interfere with each other or with the target RNA sequence. At least one of the RNA tags must interact with its binding partner in a reversible fashion.

The term “transcription cassette” as used herein refers to a nucleic acid sequence encoding a nucleic acid that is transcribed. Cassettes described herein contain multiple components such as tags, insulators and suitable restriction sites. To facilitate transcription, nucleic acid elements such as promoters, enhancers, transcriptional termination sequences and polyadenylation sequences are typically included in the transcription cassette.

The term “cellular extract” as used herein refers to proteins isolated lysated cells; for example, nuclear, cytoplasmic or organelle extracts or fractions thereof or a mixture of purified or recombinant proteins; or a combination thereof.

The term “S1” as used herein refers to the streptavidin binding sequence as DNA [SEQ ID NO:1 or SEQ ID NO:2] or RNA[SEQ ID NO: 3]

The term “2×S1” as used herein refers to the streptavidin binding sequence as DNA [SEQ ID NO: 17]

The term “MS2” as used herein refers to MS2 coat protein binding sequence as DNA [SEQ ID NO: 4] or RNA [SEQ ID NO:5].

The term “2×MS2” as used herein refers to two MS2 coat protein binding sequences as DNA [SEQ ID NO:6 and SEQ ID NO:7 and SEQ ID NO 18] or RNA [SEQ ID NO:8]

For more detailed reference of the sequences and what they are composed of:

SEQ ID NO:1—S1 DNA sequence including insulators with BglII ends

SEQ ID NO: 2

- S1 DNA sequence gaccgaccagaatcatgcaagtgcgtaagata
gtcgcgggccggg
BglII cloning site + spacers = 5′ ATCGATAAAAA and
3′ AAAAAATCGAT

SEQ ID NO:3—S1 RNA sequence

SEQ ID NO:4—MS2 DNA sequence

SEQ ID NO:5—MS2 RNA sequence

SEQ ID NO:6—2×MS2 DNA sequence including insulators with SacII ends

SEQ ID NO:7—2×MS2 DNA sequence

SEQ ID NO:8—2×MS2 RNA sequence

SEQ ID NO:9—Streptotag (streptomycin binding) tag DNA sequence with insulators and KpnI

SEQ ID NO: 10—Streptotag (streptomycin binding) tag DNA sequence

SEQ ID NO: 11—Streptotag RNA sequence

SEQ ID NO: 12—Nut (N binding) DNA sequence with insulators and KpnI ends

SEQ ID NO:13—Nut (N binding) DNA seqeunce

SEQ ID NO:14—Nut (N binding) RNA sequence. This is the RNA produced by SEQ NO 12.

SEQ ID NO:15—D8 (Sephadex binding) DNA sequence

SEQ ID NO:16—D8 RNA sequence

SEQ ID NO:17—TRAPS1 DNA—S1 tags with BglII, Cla I restriction sites and spacers.

SEQ ID NO: 18—TRAPMS2—MS2 tags with Sca I restriction sites and spacers.

SEQ ID NO: 19—TAR DNA sequence

The terminology used in the description of the invention herein is for the purpose of describing particular embodiments only, and is not intended to be limiting to the invention. As used in the description of the invention and the appended claims, the singular forms “a”, “an” and “the” are intended to include the plural forms as well, unless the context clearly indicates otherwise. Unless otherwise defined, all technical and scientific terms used herein have the same meaning as commonly understood by one of ordinary skill in the art to which this invention belongs. All publications, patent applications, patents and other references mentioned herein are incorporated by reference in their entirety.

The present invention relates to a method for isolating specific RNA-protein complexes formed in vivo. However, it can also be used to isolate or verify complexes formed in vitro. In vivo complex formation and purification is accomplished by expressing tagged versions of the RNA of interest in vivo and then using the tag to isolate associated functional RNP complexes. Tags in the form of short RNA sequences that interact with specific proteins, antibiotics or synthetic ligands can be readily inserted 5′ or 3′ to the RNA of interest (see FIG. 1A). Although a number of these potential RNA tags exist, purification with these tags gives at most a thousand-fold purification of the associated RNAs. By using two RNA tags, the TRAP-tag method of the current invention provides approximately a million-fold purification of associated RNAs, which is sufficient for the identification of most cellular proteins. The tags in the current invention must be relatively short, fully modular, and must not interfere with each other or with the RNA of interest. In addition, at least one of the tags must interact with its ligand in a reversible fashion so that RNP complexes can be eluted intact from the first ligand matrix and bound to the second matrix (see FIG. 1B). When expressed in vivo, TRAP-tagged RNAs assemble into functional complexes, and these complexes are readily purified to homogeneity.

Nucleic Acid Molecules

Functionally Equivalent Nucleic Acid Molecule or Polypeptide Sequence

The term “isolated DNA sequence” refers to a DNA sequence the structure of which is not identical to that of any naturally occurring DNA sequence or to that of any fragment of a naturally occurring DNA sequence spanning more than three separate genes. The term therefore covers, for example, (a) DNA which has the sequence of part of a naturally occurring genomic DNA molecule; (b) a DNA sequence incorporated into a vector or into the genomic DNA of a prokaryote or eukaryote, respectively, in a manner such that the resulting molecule is not identical to any naturally occurring vector or genomic DNA; (c) a separate molecule such as a cDNA, a genomic fragment, a fragment produced by reverse transcription of polyA RNA which can be amplified by PCR, or a restriction fragment; and (d) a recombinant DNA sequence that is part of a hybrid gene, i.e., a gene encoding a fusion protein. Specifically excluded from this definition are nucleic acids present in mixtures of (i) DNA molecules, (ii) transfected cells, and (iii) cell clones, e.g., as these occur in a DNA library such as a cDNA or genomic DNA library.

Modifications in the DNA sequence, which result in production of a chemically equivalent or chemically similar amino acid sequence, are included within the scope of the invention.

Modifications include substitution, insertion or deletion of nucleotides or altering the relative positions or order of nucleotides.

Sequence Identity

The invention includes modified nucleic acid molecules with a sequence identity at least about: >95% to the DNA sequences provided in SEQ ID NO: 1, SEQ ID NO 2, SEQ ID NO 4, SEQ ID NO 6, SEQ ID NO 7, SEQ ID NO 9, SEQ ID NO 10, SEQ ID NO 12, SEQ ID NO 13, SEQ ID NO 15, SEQ ID NO 17, SEQ ID NO 18, SEQ ID NO 19 (or a partial sequence thereof or their complementary sequence). Preferably about 1, 2, 3, 4, 5, 6, to 10, 10 to 25, 26 to 50 or 51 to 100, or 101 to 250 nucleotides are modified. Sequence identity is most preferably assessed by the algorithm of the BLAST version 2.1 program advanced search (parameters as above). Blast is a series of programs that are available online at http//www.ncbi.nlm.nih.gov/BLAST.

References to BLAST searches are:
Altschul, S. F., Gish, W., Miller, W., Myers, E. W. & Lipman, D. J. (1990) “Basic local alignment search tool.” J. Mol. Biol. 215:403_{—}410.
Gish, W. & States, D. J. (1993) “Identification of protein coding regions by database similarity search.” Nature Genet. 3:266_{—}272.
Madden, T. L., Tatusov, R. L. & Zhang, J. (1996) “Applications of network BLAST server” Meth. Enzymol. 266:131_{—}141.
Altschul, S. F., Madden, T. L., Schäffer, A. A., Zhang, J., Zhang, Z., Miller, W. & Lipman, D. J. (1997) “Gapped BLAST and PSI_BLAST: a new generation of protein database search programs.” Nucleic Acids Res. 25:3389_{—}3402.
Zhang, J. & Madden, T. L. (1997) “PowerBLAST: A new network BLAST application for interactive or automated sequence analysis and annotation.” Genome Res. 7:649_{—}656.

Other programs are also available to calculate sequence identity, such as Clustal W program (preferably using default parameters; Thompson, J D et al., Nucleic Acid Res. 22:4673-4680). DNA sequences functionally equivalent to the S1 SEQ ID NO: 2, or MS2 SEQ ID NO: 4 can occur in a variety of forms as described above.

The sequences of the invention can be prepared according to numerous techniques. The invention is not limited to any particular preparation means. For example, the nucleic acid molecules of the invention can be produced by cDNA cloning, genomic cloning, cDNA synthesis, polymerase chain reaction (PCR) or a combination of these approaches (Current Protocols in Molecular Biology, F. M. Ausbel et al., 1989). Sequences may be synthesized using well-known methods and equipment, such as automated synthesizers.

Hybridization

Other functional equivalent forms of the S1 SEQ ID NO: 1 and SEQ ID NO: 2 and MS2 DNA SEQ ID NO: 4 molecules can be isolated using conventional DNA-DNA or DNA-RNA hybridization techniques. These nucleic acid molecules and the S1 SEQ ID NO: 1, SEQ ID NO: 2, SEQ ID NO 17 and MS2 SEQ ID NO 4, SEQ ID NO 6, SEQ ID NO 18 sequences can be modified without significantly affecting their activity.

The present invention also includes nucleic acid molecules that hybridize to one or more of the DNA sequences provided S1 SEQ ID NO: 1, SEQ ID NO: 2, SEQ ID NO: 17 and MS2 SEQ ID NO: 4, SEQ ID NO: 6, SEQ ID NO: 18 (or a partial sequence thereof or their complementary sequence). Such nucleic acid molecules preferably hybridize to all or a portion of S1 SEQ ID SEQ ID NO: 1, SEQ ID NO: 2, SEQ ID NO: 17 or MS2 SEQ ID NO: 4, SEQ ID NO: 6, SEQ ID NO: 18 or their complement under low, moderate (intermediate), or high stringency conditions as defined herein (see Sambrook et al. (most recent edition) Molecular Cloning: A Laboratory Manual, Cold Spring Harbor Laboratory Press, Cold Spring Harbor, N.Y.; Ausubel et al. (eds.), 1995, Current Protocols in Molecular Biology, (John Wiley & Sons, NY)). The portion of the hybridizing nucleic acids is typically at least 15 (e.g. 20, 25, 30 or 50) nucleotides in length. The hybridizing portion of the hybridizing nucleic acid is at least 80% e.g. at least 95% or at least 98% identical to the sequence or a portion or all of a nucleic acid encoding S1 or S2 or their complement. Hybridizing nucleic acids of the type described herein can be used, for example, as a cloning probe, a primer (e.g. a PCR primer) or a diagnostic probe. Hybridization of the oligonucleotide probe to a nucleic acid sample typically is performed under stringent conditions. Nucleic acid duplex or hybrid stability is expressed as the melting temperature or Tm, which is the temperature at which a probe dissociates from a target DNA. This melting temperature is used to define the required stringency conditions. If sequences are to be identified that are related and substantially identical to the probe, rather than identical, then it is useful to first establish the lowest temperature at which only homologous hybridization occurs with a particular concentration of salt (e.g. SSC or SSPE). Then, assuming that 1% mismatching results in a 1 degree Celsius decrease in the Tm, the temperature of the final wash in the hybridization reaction is reduced accordingly (for example, if sequences having greater than 95% identity with the probe are sought, the final wash temperature is decreased by 5 degrees Celsius). In practice, the change in Tm can be between 0.5 degrees Celsius and 1.5 degrees Celsius per 1% mismatch. Low stringency conditions involve hybridizing at about: 1×SSC, 0.1% SDS at 50° C. High stringency conditions are: 0.1×SSC, 0.1% SDS at 65° C. Moderate stringency is about 1×SSC 0.1% SDS at 60 degrees Celsius. The parameters of salt concentration and temperature can be varied to achieve the optimal level of identity between the probe and the target nucleic acid.

The present invention also includes nucleic acid molecules from any source, whether modified or not, that hybridize to genomic DNA, cDNA, or synthetic DNA molecules that encode. A nucleic acid molecule described above is considered to be functionally equivalent to a S1 nucleic acid molecule SEQ ID NO: 1, SEQ ID NO 2, SEQ ID NO 17 of the present invention if the sequence encoded by the nucleic acid molecule is recognized in a specific manner by streptavidin and is elutable by biotin. A nucleic acid molecule described above is considered to be functionally equivalent to a MS2 SEQ ID 4, SEQ ID NO 6, SEQ ID NO 7, SEQ ID NO 18 nucleic acid molecule of the present invention if the sequence encoded by the nucleic acid molecule is recognized in a specific manner by the MS2 coat binding protein.

Vectors

The present invention provides an expression vector comprising a transcription cassette. The transcription cassette can be cloned into a variety of vectors by means that are well known in the art. Such a vector may comprise a suitably positioned restriction site or other means for insertion of a transcription cassette. The vector may also contain a selectable marker. For use in an assay or experiment, commercially available vectors such as a CMV Casper promoter vector may be employed. For use in gene therapy, vectors such as adenovirus may be employed. Cell cultures transfected or transformed with the DNA sequences of the current invention are useful as research tools particularly for studies of RNA-protein complexes. One skilled in the art will appreciate that there are a wide variety of suitable vectors.

Host Cells

A further aspect of the present invention provides a host cell containing a transcription cassette of the current invention. Examples of particularly desirable host cells include yeast, ES, P19, COS, S2 and SF9 cells. Methods known in the art for transformation, include but are not limited to electroporation, rubidium chloride, calcium chloride, calcium phosphate or chloroquine transfection, viral infection, phage transduction, microinjection, and the use of cationic lipid and lipid/amino acid complexes or of liposomes, or a large variety of other commercially available and readily synthesized transfection adjuvants, are useful to transfer the vectors of the current invention into host cells. Host cells are cultured in conventional nutrient media. The media may be modified as appropriate for inducing promoters, amplifying nucleic acid sequences of interest or selecting transformants. The culture conditions, such as temperature, composition and pH will be apparent. After transformation, transformants may be identified on the basis of a selectable phenotype.

RNA Fusion Molecules

The current invention provides for RNA fusion molecules comprising RNA tags, insulator elements and target RNA sequences. The RNA fusion molecule contains at least two different RNA tags. Suitable RNA tags include, but are not limited to streptavidin binding sequence, an MS2 coat protein binding sequence, a streptomycin binding sequence (Streptotag), a sephadex binding sequence, an N protein binding sequence, a REV binding sequence, a TAT-binding sequence and an R17 coat protein binding sequence. In some embodiments of the invention, it will be suitable to have more than one copy of an RNA tag. For example, it may be desirable to have 2×MS2 coat protein binding sequence and 2×S1 binding sequence (see FIG. 5). In another embodiment, it may desirable to have from 3× to 6×MS2 coat protein binding sequences and from 3× to 6×S1 protein binding sequences. In general, increasing the number of RNA tags in the RNA fusion molecule increases the degree of purification of the resulting RNA-protein complex due to an increase in the affinity of the RNA-protein complex for the affinity resin.

A target RNA sequence may be an oligoribonucleotide sequence or a ribonucleic acid sequence. Generally, for use in this invention, the target RNA sequence is RNA, including ribosomal RNA, RNA encoded by a gene, messenger RNA, UTRs, ribozyme RNA, catalytic RNA, small nuclear RNA, small nucleolar RNA, etc., from a microorganism, or an RNA expressed by a cell infected with a virus, or RNA from a host cell, or RNA encoded by a genomic sequence; or RNA encoded by a chemically synthesized DNA sequence or random RNA encoded by randomly isolated DNA.

Insulator elements may be placed on either side of the RNA tags and function to ensure proper folding of the RNA tags and to discourage interactions between the tags and the target RNA sequence. Examples of suitable insulator elements include, but are not limited to stretches of 4-5 identical nucleotides (eg, adenosines) coupled with paired restriction sites that do not interact with the tag or bait sequences. The 5′ and 3′ restriction sites should be identical as these sequences can then hybridize, forming a stem that forces the “insulator” polynucleotide sequences to be “unpaired” thus isolating the internal tag or bait structures from the remainder of the RNA sequences produced from a specific vector. Insulator elements may also be called spacers.

Method of Purifying

The invention provides a method for purifying an RNA-protein complex formed in vitro or in vivo. The isolated protein part of the RNA-protein complex may then be identified by various methods and techniques including but not limited to SDS-page, silver staining, Western blotting and mass spectrometry. Examples of suitable solid supports for use with the different embodiments of the current invention include affinity columns comprising bound streptavidin or bound MS2, wherein the MS2 can be bound to agarose or sepharose beads. MS2 affinity columns can also be made by crosslinking to resins such as affigel beads, or binding as a fusion protein to an appropriate resin (eg GST-MS2 to glutathione beads).

Method of Screening

The current invention relates to a method of screening for a compound that modulates or regulates the formation of an RNA-protein complex formed in vivo or in vitro. Other methods, as well as variation of the above methods will be apparent from the description of this invention. For example, the test compound may be either fixed or increased, a plurality of compounds or proteins may be tested at a single time. “Modulation”, “modulates”, and “modulating” can refer to enhanced formation of the RNA-protein complex, a decrease in formation of the RNA-protein complex, a change in the type or kind of the RNA-protein complex or a complete inhibition of formation of the RNA-protein complex. Suitable compounds that may be used include but are not limited to proteins, nucleic acids, small molecules, hormones, antibodies, peptides, antigens, cytolines, growth factors, pharmacological agents including chemotherapeutics, carcinogenics, or other cells (i.e. cell-cell contacts). Screening assays can also be used to map binding sites on RNA or protein. For example, tag sequences encoding for RNA tags can be mutated (deletions, substitutions, additions) and then used in screening assays to determine the consequences of the mutations.

Kits

The invention includes kits for detecting RNA-protein complexes comprising at least one isolated DNA construct of the invention or at least one vector of the current invention.

Tandem RNA Purification

A number of RNA motifs suitable as RNA affinity tags exist. We first tested five of these for potential use in our double-tagging system. These include the “streptotag”, a streptomycin binding aptamer (Bachler et al., 1999), “S1”,a streptavidin binding aptamer (Srisawat and Engelke, 2001), “D1”, a sephadex binding aptamer (Srisawat et al., 2001), the MS2 phage coat protein binding RNA (Jurica et al., 2002), “TAR”, a Tat protein binding sequence (Puglisi et al., 1995) and the lambda phage box B RNA (Lazinski et al., 1989). Table 1 shows the relative binding and elution efficiencies of each ³²P-labeled tag and its ligand. Two of the five tags, the streptavidin (S1, SEQ ID NO: 1 and SEQ ID NO: 2) and MS2 coat protein (MS2) tags, were found to bind and elute efficiently under the desired purification conditions. Importantly, neither tag cross-reacted with any of the other tested ligands.

Greater than 95% of the S1 tag SEQ ID NO: 1 and SEQ ID NO: 2 bound to streptavidin agarose beads, of which 95% could be recovered with the addition of biotin. Approximately 75% of the loaded MS2 tag bound to GST-coat protein-beads, and approximately 70% of the loaded tag could be eluted with glutathione.

TABLE 1
RNA aptamer tags tested for use in TRAP vectors.

| RNA | | Length | | | | |
|---|---|---|---|---|---|---|
| aptamer | SEQ ID NO | (nucleotides) | Affinity target | Eluted with: | % Bound | % Elu [symbol] |
| Streptotag | 9-DNA | 64 | 8-hydroxy- | Streptomycin | 21% ± 2% | 12% ± [symbol] |
| | 11-RNA | | streptomycin | | | |
| MS2, | 4,6-DNA | 38, 96 | Coat Binding | Reduced | 73% ± 3% | 68% ± [symbol] |
| 2 × MS2 | 5,8-RNA | | Protein | Glutathione | | |
| S1 | 1-DNA | 68 | Streptavidin | Biotin | >99% | 94% ± [symbol] |
| | 3-RNA | | | | | |
| D8 | 15-DNA | 64 | Sephadex | n/a | 34% ± 1% | 21% ± [symbol] |
| | 16-RNA | | | | | |
| TAR | 19-DNA | 29 | Tat Protein | Tat Peptide | 80% ± 5% | ND |
| Nut 1-39 | 12-DNA | 33 | N-protein 1-22 | n/a | <1% | <1% |
| | 14-RNA | | | | | |

Next the ability of the Streptavidin and MS2 coat protein tags to function together and in the presence of an RNA target molecule was tested. Cassettes containing a T7 promoter, the two RNA tags, alternative target RNA insertion sites and a poly A tail were made (FIG. 1B). Insulator elements, consisting of 8-10 Adenosines flanked by identical restriction sites, were placed on either side of each tag to ensure proper folding of the tags and to discourage interactions between the tags and the inserted target RNA. ³²P-labeled RNAs were first tested for retention and elution on streptavidin and GST-coat protein columns. Both tags worked with much the same efficiency as when used individually. A construct containing 2×S1 tag SEQ ID NO: 17 and 2×MS2 tags SEQ ID NO: 18 are preferred.

TRAP Tag Purification Using in Vitro Transcribed RNA

Next the constructs were tested for the ability to purify specific RNA binding proteins from a complex protein mixture. Two, approximately 100-nucleotide long elements from the Drosophila wingless gene mRNA (WLE1 and WLE2) were chosen for this purpose. These elements are required for the asymmetrical localization of wingless transcripts to apical cytoplasm (Simmonds et al., 2001). The two elements show no similarity in sequence or predicted secondary structure and exhibit marked differences in their ability to localize transcripts. On the other hand, both appear to mediate localization via dynein-dependent microtubule transport (Wilkie and Davis, 2601). Hence, they probably interact with unique but overlapping subsets of proteins.

The tagged RNAs were expressed in vitro, and the cold RNA mixed for 30 minutes with Drosophila embryo extracts prior to purification over the two columns. FIG. 2A shows that each of the tagged localization elements did indeed associate with different subsets of proteins that were not bound by beads or tags alone. Nine (9) of nineteen (19) proteins identified by Mass spectrometry are known or predicted RNA binding proteins (Simmonds and Krause, in preparation). FIG. 2B shows that one of these proteins is Bic-D, a protein previously implicated as being required for apical mRNA transport in blastoderm stage Drosophila embryos (Bullock and Ish-Horowicz, 2001).

Localization of TRAP-Tagged WLE RNAs in Drosophila Embryos

The final test was to ensure that complexes formed on the tagged RNAs in vivo are both active and readily purified. To confirm this, tagged WLE constructs were first fluorescently labeled and injected into syncitial blastoderm stage embryos. RNAs with an apical localization motif will move from the site of injection upwards, between the syncitial nuclei to the apical surface (Bullock and Ish-Horowicz, 2001). FIG. 3A shows untagged WLE2 RNA after localization to the apical surface. FIG. 3C shows that TRAP-tagged WLE2 RNA localizes to the apical surface in an indistinguishable fashion. Thus, the tags appear to have no effect on the function of the localizing element. TRAP-tagged wingless localization elements expressed in transgenic embryos also localized apically (data not shown). Extracts were made from these transgenic fly lines and used for purification of WLE-associated proteins.

TRAP Tag Purification Using RNA Expressed in Vivo.

FIG. 4 shows that, as in vitro, each of the tagged WLE constructs binds a different subset of proteins. The identities of some of these proteins were determined by Mass Spectrometry. Once again, one of the purified proteins included Bic-D.

Note that, although the proteins identified here were easily detected using a small amount of extract and silver staining, the reversibility of the two columns permits the optional use of a second round of purification to detect proteins of very low abundance and proteins that do not bind the bait stoichiometrically or in all cell types. The S1 tag SEQ ID NO:1 SEQ ID NO: 2 is particularly well suited for repeated rounds of purification. It provides high degrees of purification with little loss of material, and the biotin used for elution is easily removed. Biotin removal is achieved by running the eluate over an avidin column (the S1 tag SEQ ID NO:1 SEQ ID NO: 2 does not bind avidin). The flow-through is then bound to the second streptavidin column and eluted with biotin as before. This approach can also be used for prior removal of streptavidin binding proteins, should they be present in extracts in large amounts. Clearly, this approach is applicable to any cell or tissue type. The TRAP cassette is simply placed into an appropriate vector. Although the in vivo application of the method is the most powerful version of this approach, in vitro assays are also clearly applicable. For example, using mutagenesis, the importance of specific nucleotides and structural aspects of known or newly discovered interactions can be rapidly tested with in vitro expressed RNAs and then confirmed in vivo. This approach is also amenable to high throughput analyses. This is particularly true for in vitro work with extracts, and with transfected or virally infected cells. With a little more effort, the approach can also be applied to transformed cells and transgenic tissues. For example, as has been done for proteins in yeast, TRAP tags could be placed within each yeast gene and substituted for the endogenous gene by homologous recombination. However, this approach is probably the most useful for small RNAs and functionally characterized RNA motifs. It is also possible to identify other RNAs bound within TRAP-purified complexes. This can be achieved either by RTPCR, or more globally by labeling the RNAs and hybridizing to microarrays.

Given the rapidly growing number of important processes controlled by RNAs and the proteins that bind them, TRAP-tagging should prove to be a key tool in the elucidation of these functions on a genomic scale. Once well characterized, functional RNA elements can serve as drug targets (RNAi etc). Viral RNAs such as HIV, hepatitis B, and the proteins that bind them, are particularly applicable targets. Examples of such uses include the treatment of viral infections, the control of cellular proliferation and the stimulation of neuronal regeneration.

Vector Construction

Initial TRAP vectors were constructed using a cassette-based approach to allow for maximum versatility. Cassettes were made using paired oligonucleotides cloned into pSP72 (Promega). To facilitate further cloning into other expression vectors the plasmid was modified by addition of an SpeI restriction site 3′ to the polylinker XhoI site using the paired olgionucleotides 5′SpeI TCGAGACTAGT and 3′SpeI AGCTTGATCAG.

The streptavidin aptamer was added by hybridization of S1BglII5′ (ATCTAAAAGACCGACCAGAATCATGCAAGTGCGTAAGATAGTCGCGGGCCGGGAAAAAA and S1BglII3′ (ATC TTTTTTCCCGGCCCGCGACTATCTTACGCACTTGCATGATTCTGGTCGGTCTTTTTA) oligonucleotides and insertion into the BglII site of pSP72. (see FIG. 5)

The MS2 aptamer was created by hybridizing the oligos MS2 5′ (CAAACGACTCTAGAAAACATGAGGATCACCCATGTCTGCAGG) and MS2 3′ (TCGACCTGCAGACATGGGTGATCCTCATGTTTTCTAGAGTCGTTTTCTGAGC) and the oligos MS2 5′ (TCGACTCTAGAAACATGAGGATCACCCATGTCTGCAGGTCAAAAAGAGCT) and MS2 3′ (CTTTTTGACCTGCAGACATGGGTGATCCTCATGTTTTCTAGAG), subcloning the two fragments separately into pBluescript SK⁻ (Stratagene) and were then ligating the excised fragments together with the pSP72 vector linearized with SacII. Clones were then sequenced to identify those with MS2 aptamer sequences in the correct orientation. Primers used to create other tags tested include 5′Streptotag KpnI (CAAAAGGATCGCATTTGGACTTCTGCCCAGGGTGGCACCACGTGCGGATCCAAAAGGTAC), 3′Streptotag KpnI (CTTTTGGATCCGACCGTGGTGCCACCCTGGGCAGAAGTCCAAATGCGATCCTTTTGGTAC), N-5′KpnI (GATCCTTTTCGGGTGAAAAAGGGCTTTTG) ad N3′KpnI (GATCCAAAAGCCCTTTTTCAGGGCAAAG). Plasmids produced by these manipulations are referred to respectively as pTRAPS1, pTRAPMS2, pTRAPS1MS2, pTRAPN, pTRAPS1N. The wingless 3′UTR regions referred to as WLE1 (wg 3′UTR 1-181), WLE2 (wg 3′UTR 659-773), 2×WLE2 (tandem duplication of WLE2), WLE2-mutated (WLE2 with residues 678-689 mutated to the sequence AGATCT) and wg 3′UTR 360-1107 were amplified by polymerase chain reaction (PCR) and cloned into the BamHI site of the pTRAPS1MS2 vector to create the vectors pTRAPS1MS2+WLE1, pTRAPS1MS2+WLE2, pTRAPS1MS2+2×WLE2 pTRAPS1MS2+WLE2(mutated) and pTRAPS1MS2+wg 3′UTR 360-1107 respectively. For constructs that could be expressed in transgenic flies, HpaI-SpeI fragements of pTRAPS1MS2+WLE1, pTRAPS1MS2+WLE2, pTRAPS1MS2+wg 3′UTR 360-1107, pTRAPS1MS2+2×WLE2, pTRAPS1MS2+WLE2(mutated) or pTRAPS1MS2 (no insert) were subcloned into BglII-StuI cut pCASPER-HS (Thummel and Pirrotta 1992). The resulting vectors, pCASPER-TRAPWLE1, pCASPER-TRAPWLE2, pCASPER-TRAP2×WLE2, pCASPER-TRAPWLE2 (mutated) and pCASPER-TRAP, were introduced into Drosophila embryos by P-element-mediated transformation (Spradling and Rubin 1982). A minimum of three independent transgenic lines was isolated for each construct injected.

Production of GST-MCP Beads

A coat protein GST fusion was made by subcloning a PCR fragment consisting of the entire open reading frame, with a BamHI site added 3′ and an XhoI site added 5′, into the vector pGEX4T-1 (Pharmacia). The fusion protein was expressed in E. coli BL21 cells grown at 37° C. for 3 hours (OD₆₀₀ of 1.8) and then induced with 100 mM IPTG for 4.5 hours. Cells were pelleted in 250 ml aliquots, quick frozen in liquid nitrogen and stored for as long as 2 months at −70° C. Cell pellets were lysed by sonication (5 min at 50%) and bound to Glutathione-Sepharose beads (Pharmacia) as specified by the manufacturer. After extensive washing, the fusion protein was cross-linked to the beads using 20 mM dimethyl pimelimidate dissolved in 200 mM HEPES (pH 8.5) buffer (Bar-Peled et al., 1996). The cross-linked affinity resin can be stored for at least 6 months at −20° C. in storage buffer (HEPES pH 7.4, 80 mM NaCl, 1 mM EDTA, 1 mM DTT, 40% glycerol). Alternatively, if glutathione elution from the coat protein beads is desired, the protein can be left uncoupled. However, the eluted protein may then obscure the presence of other specifically bound proteins.

In Vitro RNA Expression

Templates for transcription were made by linearization of pTRAP constructs with XhoI, phenol/chloroform extraction to remove the enzyme and ethanol precipitation. 25 μl transcription reactions contained 1 μg linearized pTRAP DNA, 5 μl 5× T7 RNA polymerase buffer (400 mM Tris-HCl pH 8.0, 60 mM MgCl₂), 5 μl 10 mM NTP mix, 1 μl 0.75 mM DTT (RNAse free), 20U placental RNAse inhibitor (MBI), 15U T7 RNA polymerase and RNAse free water to 25 μl. Reactions were incubated at 37° C. for 2 hours, the resulting RNA precipitated using 0.4M LiCl and 2.5 volumes of ethanol and the pellets resuspended in 40 μl RNAse free water (Ambion). The yield of RNA product is approximately 25 μg, which is the amount of RNA added to 1 ml of Drosophila cytoplasmic extract (described below).

Extract Preparation

Drosophila embryos were collected for 4 or 12 hours and aged an additional 4 hours. TRAP constructs in transgenic embryos were induced using a 30 min heat pulse (36.5° C.). Cytoplasmic extracts were prepared essentially as described by Moritz (Sullivan et al., 2000) with the following changes. TRAP Purification Buffer (5× TPB stock solution=300 mM HEPES pH 7.4, 50 mM MgCl, 400 mM NaCl, 0.5% Triton X-100) was used for all steps of extract production. TPB working solution was made by adding glycerol to 10%, proteinase inhibitor (Complete EDTA free; Roche) and DTT (1 mM final) to diluted stock solution. Dechorionated embryos were washed twice in the dounce with 3 volumes of TPB buffer and then removing all but enough buffer to just cover the embryos. Homogenized extract was passed just once through miracloth. The resulting filtrate was spun at 14,000 g for 10 minutes (in microfuge or appropriate centrifuge tubes, depending on volume) and transferred to new tubes. Centrifugation was repeated as necessary until the filtrate was clear. If not used right away, glycerol was added to 20% final, and the extract flash frozen and stored at −70C.

TRAP Purification

RNAse free conditions and solutions made with DEPC treated water were used throughout.

For in vitro purifications, thawed lysate was re-centrifuged for 5 min at 14,000 g, and 10 μg RNA added per ml of lysate. After incubation for 2-3 hours at 4° C., the lysate was mixed with streptavidin agarose beads (Sigma: 200 μl beads/ml extract) pre-equilibrated 1×TPB solution. After gentle rocking for 1 h at 4° C., the mixture was added to an RNAse-free chromatography column and allowed to settle. Columns were then un-plugged, the unbound material allowed to flow-through and then washed three times with 1 ml TPB. Bound complexes were eluted by plugging the columns, adding 500 μl Biotin elution buffer, (1×TPB+5 mM d-Biotin, Sigma), incubating for 1 hr at 4° C. and then opened and the eluate collected. An additional 250 μl Biotin elution buffer was added to the column and the eluates pooled. An option at this point is to repeat the streptavidin affinity chromatography after first removing the biotin (using Avidin-agarose beads).

Streptavidin eluates were then bound to GST-MCP beads. Approximately 50 μl of GST-CP sepharose beads, pre-washed 3 times in 1×TPB, was used per 500 μl of streptavidin eluate. After rocking for 1 h at 4° C., the mixture was transferred to a plugged RNAse-free mini column. After the beads settled, the column was unplugged, the unbound material allowed to flow-through and the beads washed three times with 1 ml 1×TPB. Bound complexes were eluted using either glutathione elution buffer (Pharmacia), high salt (5×TPB), RNAse (200 μl of 2 mg/mlRNAseA+5000 u/mlRNAse T1 (Fermentas) or various denaturants (eg. urea, SDS). This was done by adding one bed volume of elution buffer, incubating for 30 min, eluting, rinsing three times with elution buffer and pooling the four eluates. Proteins were then resolved by SDS PAGE and identified by Trypsin proteolysis, Mass Spectrometry (Fenyo 1998) and submission of the data to Drosophila genomic databases (Adams 2000).

### REFERENCES

Adams, M. D. et al. The genome sequence of Drosophila melanogaster. Science 287, 2185-2195 (2000).
Bachler, M., Schroeder, R. & von Ahsen, U. StreptoTag: a novel method for the isolation of RNA-binding proteins. RNA 5, 1509-1516 (1999).
Bar-Peled, M., and N. V. Raikhel. A method for isolation and purification of specific antibodies to a protein fused to the GST. Anal. Biochem. 241:140-142 (1996)
Bell, L. R., Maine, E. M., Schedl, P. & Cline, T. W. Sex-lethal, a Drosophila sex determination switch gene, exhibits sex-specific RNA splicing and sequence similarity to RNA binding proteins. Cell 55, 1037-1046 (1988).
Berkhout, B., Silverman, R. H. & Jeang, K. T. Tat trans-activates the human immunodeficiency virus through a nascent RNA target. Cell 59, 273-282 (1989).
Bullock, S. L. & Ish-Horowicz, D. Conserved signals and machinery for RNA transport in Drosophila oogenesis and embryogenesis. Nature 414, 611-616 (2001).
Chartrand, P., Singer, R. H. & Long, R. M. RNP localization and transport in yeast. Annu Rev Cell Dev Biol 17, 297-310 (2001).
Doudna, J. A. & Rath, V. L. Structure and function of the eukaryotic ribosome: the next frontier. Cell 109, 153-156 (2002).
Dreyfuss, G., Kim, V. N. & Kataoka, N. Messenger-RNA-binding proteins and the messages they carry. Nat Rev Mol Cell Biol 3, 195-205 (2002).
Erdmann, V. A., Barciszewska, M. Z., Hochberg, A., de Groot, N. & Barciszewski, J. Regulatory RNAs. Cell Mol Life Sci 58, 960-977 (2001).
Fenyo, D., Qin, J. & Chait, B. T. Protein identification using mass spectrometric information. Electrophoresis 19, 998-1005 (1998).
Jurica, M. S., Licklider, L. J., Gygi, S. R., Grigorieff, N. & Moore, M. J. Purification and characterization of native spliceosomes suitable for three-dimensional structural analysis. RNA 8,426-439 (2002).
Keene, J. D. Ribonucleoprotein infrastructure regulating the flow of genetic information between the genome and the proteome. Proc Natl Acad Sci USA 98, 7018-7024 (2001).
Lazinski, D., Grzadzielska, E. & Das, A. Sequence-specific recognition of RNA hairpins by bacteriophage antiterminators requires a conserved arginine-rich motif. Cell 59, 207-218 (1989).
Le, S., Stemglanz, R. & Greider, C. W. Identification of two RNA-binding proteins associated with human telomerase RNA. Mol Biol Cell 11, 999-1010 (2000).
Lee, J. T. & Jaenisch, R. Long-range cis effects of ectopic X-inactivation centres on a mouse autosome. Nature 386, 275-279 (1997).
Mahalingam, S., Meanger, J., Foster, P. S. & Lidbury, B. A. The viral manipulation of the host cellular and immune environments to enhance propagation and survival: a focus on RNA viruses. J Leukoc Biol 72, 429-439 (2002).
Meller, V. H. et al. Ordered assembly of roX RNAs into MSL complexes on the dosage-compensated X chromosome in Drosophila. Curr Biol 10, 136-143 (2000).
Pesole, G. et al. Structural and functional features of eukaryotic mRNA untranslated regions. Gene 276, 73-81 (2001).
Puglisi, J. D., Chen, L., Blanchard, S. & Frankel, A. D. Solution structure of a bovine immunodeficiency virus Tat-TAR peptide-RNA complex. Science 270, 1200-1203 (1995).
Salido, E. C., Yen, P. H., Mohandas, T. K. & Shapiro, L. J. Expression of the X-inactivation-associated gene XIST during spermatogenesis. Nat Genet 2, 196-199 (1992).
Simmonds, A. J., dosSantos, G., Livne-Bar, I. & Krause, H. M. Apical localization of wingless transcripts is required for Wingless signaling. Cell 105, 197-207 (2001).
Spradling, A. C. & Rubin, G. M. Transposition of cloned P elements into Drosophila germ line chromosomes. Science 218, 341-347 (1982).
Srisawat, C. & Engelke, D. R. Streptavidin aptamers: affinity tags for the study of RNAs and ribonucleoproteins. RNA 7, 632-641 (2001).
Srisawat, C., Goldstein, I. J. & Engelke, D. R. Sephadex-binding RNA ligands: rapid affinity purification of RNA from complex RNA mixtures. Nucleic Acids Res 29, E4 (2001).
Swan, A. & Suter, B. Role of Bicaudal-D in patterning the Drosophila egg chamber in mid-oogenesis. Development 122, 3577-3586 (1996).
Szymanski, M., Barciszewska, M. Z., Zywicki, M. & Barciszewski, J. Noncoding RNA transcripts. J Appl Genet 44, 1-19 (2003).
Thummel, C. & Pirrotta, V. Technical notes: new pCasper P-element vectors. Drosophila Information Service 71, 150 (1992).
Tenenbaum, S. A., Carson, C. C., Lager, P. J. & Keene, J. D. Identifying mRNA subsets in messenger ribonucleoprotein complexes by using cDNA arrays. Proc Natl Acad Sci U S A 97, 14085-14090 (2000).
Wilkie, G. S. & Davis, I. Drosophila wingless and pair-rule transcripts localize apically by dynein-mediated transport of RNA particles. Cell 105, 209-219 (2001).

We claim:

1. A method for purifying an RNA-protein complex formed in vitro comprising:
(a) providing an RNA fusion molecule comprising a target RNA sequence and at least two different RNA tags, wherein at least one RNA tag interacts with a ligand in a reversible manner;
(b) contacting the RNA fusion molecule with a cellular extract;
(c) providing conditions that allow the formation of an RNA-protein complex on the target RNA sequence; and
(d) subjecting the RNA-protein complex to at least two different affinity purification steps, each step comprising binding one RNA tag to an affinity resin capable of selectively binding one RNA tag and eluting the RNA tag from the affinity resin after substances not bound to the affinity resin have been removed.

2. A method for purifying an RNA-protein complex formed in vitro comprising:
(a) providing an RNA fusion molecule comprising a target RNA sequence and at least two different RNA tags, wherein at least one RNA tag interacts with a ligand in a reversible manner;
(b) contacting the RNA fusion molecule with a protein mixture;
(c) providing conditions that allow the formation of an RNA-protein complex on the target RNA sequence; and
(d) subjecting the RNA-protein complex to at least two different affinity purification steps, each step comprising binding one RNA tag to an affinity resin capable of selectively binding one RNA tag and eluting the RNA tag from the affinity resin after substances not bound to the affinity resin have been removed.

3. A method for purifying an RNA-protein complex formed in vivo comprising:
(a) expressing in a eukaryotic cell an RNA fusion molecule comprising a target RNA sequence and at least two different RNA tags, wherein at least one RNA tag interacts with a ligand in a reversible manner;
(b) providing conditions that allow the formation of an RNA-protein complex on the target RNA sequence;
(c) generating a cellular extract;
(d) subjecting the cellular extract to at least two different affinity purification steps, each step comprising binding one RNA tag to an affinity resin capable of selectively binding one RNA tag and eluting the RNA tag from the affinity resin after substances not bound to the affinity resin have been removed.

4. The method of claim 1, 2, or 3 wherein at least one RNA tag is repeated.

5. The method of claim 1, 2, 3, or 4 wherein the RNA tags are selected from the group consisting of a streptavidin binding sequence (S1), a MS2 coat protein binding sequence, a streptomycin binding sequence (Streptotag), a sephadex binding sequence (D8), a N protein binding sequence (nut), a REV binding sequence, a TAT-binding sequence and a R17 coat protein binding sequence.

6. The method of claim 5, wherein the RNA tags comprise at least one streptavidin binding sequence and at least one MS2 coat protein binding sequence.

7. The method of claim 1, 2, 3, 4, 5, or 6 wherein at least one RNA tag binds to an affinity resin through a fusion protein comprising:
(a) a polypeptide that binds specifically to the RNA tag; and
(b) a polypeptide that binds specifically to the affinity resin.

8. The method of claim 7 wherein the polypeptide that binds specifically to the affinity resin is selected from the group consisting of a maltose binding protein, a 6-histidine peptide, glutathione S transferase and a portion thereof sufficient to bind specifically to the affinity resin.

9. The method of claim 1, 2, 3, 4, 5, 6, 7, or 8, wherein the RNA fusion molecule further comprises at least one insulator sequence.

10. An RNA fusion molecule comprising:
(a) a target RNA sequence; and
(b) at least two different RNA tags, wherein at least one RNA tag interacts with a ligand in a reversible fashion.

11. The RNA fusion molecule of claim 10, wherein at least one RNA tag is repeated.

12. The RNA fusion molecule of claim 10 or 11, wherein the RNA tags are selected from the group consisting of a streptavidin binding sequence (S1), a MS2 coat protein binding sequence, a streptomycin binding sequence (Streptotag), a sephadex binding sequence (D8), a N protein binding sequence (nut), a REV binding sequence, a TAT-binding sequence and a R17 coat protein binding sequence.

13. The RNA fusion molecule of claim 12, wherein the RNA tags comprise at least one streptavidin binding sequence and at least one MS2 coat protein binding sequence.

14. The RNA fusion molecule of claim 9, 10, 11, 12 or 13 further comprising at least one insulator sequence.

15. An isolated DNA construct encoding the RNA fusion molecule of claim 9, 10, 11, 12, 13 or 14.

16. A vector comprising the isolated DNA construct of claim 15.

17. A host cell comprising the vector of claim 16.

18. A method for screening a test compound for its ability to modulate an RNA-protein complex comprising:
(a) performing the method according to claim 1;
(b) performing the method according to claim 1, wherein the cellular extract further comprises a test compound; and
(c) observing a difference, if any, between the RNA-protein complex purified in step (a) and the RNA-protein complex, if any, purified in step (b), wherein the presence of the difference indicates that the test compound modulates the RNA-protein complex.

19. A method for screening a test compound for its ability to modulate an RNA-protein complex comprising:
(a) performing the method according to claim 2;
(b) performing the method according to claim 2, wherein the cellular extract further comprises a test compound; and
(c) observing a difference, if any, between the RNA-protein complex purified in step (a) and the RNA-protein complex, if any, purified in step (b), wherein the presence of the difference indicates that the test compound modulates the RNA-protein complex.

20. A kit for detecting an RNA-protein complex comprising the RNA fusion molecule of claim 9, 10, 11, 12, 13 or 14.

21. A kit for detecting an RNA-protein complex comprising the isolated DNA construct of claim 15.

22. A kit for detecting an RNA-protein complex comprising the vector of claim 16.


SEQUENCE LISTING (19 sequences)

SEQ ID NO: 1 - DNA, 68 bp, Artificial Sequence
  S1 DNA sequence, including insulators with BglII ends
atcgataaaaagaccgaccagaatcatgcaagtgcgtaagatagtcgcgggccgggaaaaaaatcgat

SEQ ID NO: 2 - DNA, 45 bp, Artificial Sequence
  S1 DNA sequence, BglII cloning site and spacers
gaccgaccagaatcatgcaagtgcgtaagatagtcgcgggccggg

SEQ ID NO: 3 - RNA, 68 nt, Artificial Sequence
  S1 RNA sequence
aucgauaaaaagaccgaccagaaucaugcaagugcguaagauagucgcgggccgggaaaaaaaucgau

SEQ ID NO: 4 - DNA, 38 bp, Artificial Sequence
  MS2 DNA sequence
gactctagaaacatgaggatcacccatgtctgcaggtc

SEQ ID NO: 5 - RNA, 38 nt, Artificial Sequence
  MS2 RNA sequence
gacucuagaaacaugaggaucacccaugucugcagguc

SEQ ID NO: 6 - DNA, 96 bp, Artificial Sequence
  two MS2 DNA sequences, including insulators with SacII ends
gagctcaaaaacgactctagaaacatgaggatcacccatgtctgcaggtcgactctagaaacatgaggataccatgtctgcaggtcaaaagagctc

SEQ ID NO: 7 - DNA, 75 bp, Artificial Sequence
  two MS2 DNA sequences
cgactctagaaacatgaggatcacccatgtctgcaggtcgactctagaaacatgaggataccatgtctgcaggtc

SEQ ID NO: 8 - RNA, 96 nt, Artificial Sequence
  two MS2 RNA sequences
gagcucaaaaacgacucuagaaacaugaggaucacccaugucugcaggucgacucuagaaacaugaggauaccaugucugcaggucaaaagagcuc

SEQ ID NO: 9 - DNA, 64 bp, Artificial Sequence
  Streptotag DNA sequence, including insulators and KpnI
gtaccaaaaggatcgcatttggacttctgcccagggtggcaccacgtgcggatccaaaaggtac

SEQ ID NO: 10 - DNA, 46 bp, Artificial Sequence
  Streptotag DNA sequence
ggatcgcatttggacttctgcccagggtggcaccacgtgcggatcc

SEQ ID NO: 11 - RNA, 64 nt, Artificial Sequence
  Streptotag RNA sequence
guaccaaaaggaucgcauuuggacuucugcccaggguggcaccacgugcggauccaaaagguac

SEQ ID NO: 12 - DNA, 33 bp, Artificial Sequence
  Nut DNA sequence, including insulators and KpnI ends
gatccttttcgggtgaaaaagggcttttggtac

SEQ ID NO: 13 - DNA, 15 bp, Artificial Sequence
  Nut DNA sequence
cgggtgaaaaagggc

SEQ ID NO: 14 - RNA, 33 nt, Artificial Sequence
  Nut RNA sequence produced from SEQ NO 12
gauccuuuucgggugaaaaagggcuuuugguac

SEQ ID NO: 15 - DNA, 64 bp, Artificial Sequence
  D8 DNA sequence
ccgaccagaagtccgagtaatttacgttttgatacggttgcggaacttgctatgtgcgtctaca

SEQ ID NO: 16 - RNA, 64 nt, Artificial Sequence
  D8 RNA sequence
ccgaccagaaguccgaguaauuuacguuuugauacgguugcggaacuugcuaugugcgucuaca

SEQ ID NO: 17 - DNA, 139 bp, Artificial Sequence
  TRAPS1 DNA, including BglII, ClaI restriction sites and spacers
agatctaaaagaccgaccagaatcatgcaagtgcgtaagatagtcgcgggccgggaaaaaagatctgatatcatcgataaaaagaccgaccagaatcatgcaagtgcgtaagatagtcgcgggccgggaaaaaatcgat

SEQ ID NO: 18 - DNA, 102 bp, Artificial Sequence
  TRAPMS2 DNA, including ScaI restriction site and spacers
gagctcaaaaacgactctagaaaacatgaggatcacccatgtctgcaggtcgactctagaaaacatgaggatcacccatgtctgcaggtcgaaaaagagctc

SEQ ID NO: 19 - DNA, 44 bp, Artificial Sequence
  TAR DNA sequence
agatctaaaagtcgtgtagctcattagctccgacaaaaagatct